(19) United States
(12) Patent Application Publication
(10) Pub. No.: US 2006/0234374 A1
(43) Pub. Date: Oct. 19, 2006

(54) Malignant Cells and Method for Selecting the Same

(76) Inventor: Pranela Rameshwar, Maplewood, NJ (US)

Correspondence Address: LICATA & TYRRELL P.C., 66 E. MAIN STREET, MARLTON, NJ, 08053, US

(21) Appl. No.: 11/279,420

(22) Filed: Apr. 12, 2006

Related U.S. Application Data
(60) Provisional application No. 60/670,808, filed on Apr. 13, 2005.

(51) Int. Cl. C12N 5/00; C12N 5/02

(52) U.S. Cl. 435/325

(57) ABSTRACT

The present invention is a method for selecting an enriched population of malignant cells. It has been found that by depleting fibroblasts from a population of cells obtained from a tissue sample; selecting epithelial cells from the fibroblast-depleted population; and culturing the selected epithelial cell population in the presence of bone marrow stromal cells, an enriched population of malignant cells can be selected. In particular, when the malignant cells are constructively passaged on the bone marrow stromal cells, anchorage-independent cells can be obtained. Advantageously, the instant method provides malignant cells which exhibit a preference for bone marrow stromal cells.

This application claims benefit of U.S. Provisional Patent Application Ser. No. 60/670,808, filed Apr. 13, 2005, the contents of which is incorporated hereby by reference in its entirety.

This invention was made in the course of research sponsored by the National Cancer Institute (Grant No. CA89868). The U.S. government may have certain rights in this invention.

### BACKGROUND OF THE INVENTION

Breast cancer metastasis to the bone marrow correlates with poor prognosis (Mansi, et al. (1989) J. Clin. Oncol. 7:445-449). Seemingly curative therapies in patients with both metastatic and non-metastatic breast cancer have recurrence of breast cancer cells from the bone marrow (Mansi, et al. (1989) supra). Such resurgence can occur even twenty years after remission (Mansi, et al. (1989) supra; Gluck (1995) Can. J. Oncol. 1:58-62). Based on these reports; it is believed that breast cancer cells entering and surviving in bone marrow are either subsets of breast cancer cells with unique properties, or are located in an area which protects them from treatment modalities. The ability of breast cancer cells resurging from the bone marrow is consistent with preference for this organ (Gluck (1995) supra) and the ability of bone marrow to maintain survival of growth-arrested cancer cells (Korah, et al. (2004) Cancer Res. 64:4514-4522). Since resurgence occurs even in early stages of breast cancer, it appears breast cancer cells may enter bone marrow long before the tumor can be detected by conventional clinical methods.

Bone marrow involvement in breast cancer metastasis is evident from the interaction between stromal cell-derived factor 1α (SDF-1α) and CXCR4 (Muller, et al. (2001) Nature 410:50-56). Malignant breast tissue cells, unlike those from healthy tissue, express high levels of CXCR-4. Since stromal cells produce SDF-1α (Bonnet (2002) J. Path. 197:430-440), breast cancer cells expressing CXCR4 are suggested to be attracted and retained in organs that express SDF-1α, such as bone marrow, lung, and liver (Muller, et al. (2001) supra). The density of CXCR-4 on breast cancer cells is proportional to the invasiveness of the cancer (Kato, et al. (2003) Breast Can. Res. 5:R144-R150). Recent studies with mice show that there may be two subtypes of breast cancer cells that enter the bone marrow (Rao, et al. (2004) Can. Res. 64:2874-2881). For example, breast cancer cells with high proliferative potential are found within the central/cellular areas of the bone marrow cavity (Rao, et al. (2004) supra). Another breast cancer cell subset with long doubling times is located close to the endosteum (Rao, et al. (2004) supra). Moreover, bone marrow stromata isolated from carcinoma patients has been suggested to facilitate the growth and release of carcinoma cells into the systemic circulation, as evidenced by lack of adhesion of MCF-7 cells to bone marrow stromata isolated from carcinoma patients (Nicola, et al. (2003) Clin. Exp. Metast. 20:471-479). Thus, defining the properties of cancer cell subtypes in the bone marrow and understanding the mechanisms involved in cancer cell entry in the bone marrow are key to improving cancer treatment and eradication.

While studies using cancer cell lines with varying degrees of metastatic potentials serve as model systems in the majority of research laboratories, cancer cell lines may not always provide relevant information. Some cell lines have been distributed within laboratories so that relevant information regarding their source and number of passages are lost. This leads to heterogeneity within a particular line and to seemingly irreproducible data amongst laboratories. Another disadvantage of cancer cell lines is their genomic instability. Cancer cell lines subjected to multiple passages generally results in a population of cells with varying degrees of tumorigenic potential. Thus, cancer cell lines can provide inconsistent results, and may not accurately represent the behavior of cancer cells in vivo.

In addition to cell line heterogeneity and genomic instability, cancer cell lines can be phenotypically divergent from cancer cells in vivo. For example, while numerous components of the ubiquitin/proteasome pathway have been shown to be upregulated in breast cancer cells isolated from breast cancer patients, the ubiquitin/proteasome pathway is not activated in the well-characterized MCF10 breast cancer cell line (Chen & Madura (2005) Cancer Res. 65:5599-5606).

As cancer cell lines can exhibit phenotypes divergent from that of cancer cells in vivo, it is essential to use primary cancer cells in experiments to mimic in vivo behavior of cancer cells. However, while primary cancer cells are desirable, there are limitations as to their availability. Surgical tissues are generally small and clinical analyses of excised tissue is of primary importance, leaving little or no sample for experimental analyses. This is most evident when samples are obtained by non-invasive methods such as needle biopsies. Another limitation is the difficulty in separating malignant and normal cells from surgical tissues.

Thus, there is a need in the art for a reliable method for selecting and maintaining primary malignant cells from a small tissue sample to facilitate drug discovery and cancer prevention. The present invention meets this need in the art.

### SUMMARY OF THE INVENTION

The present invention is a method for selecting an enriched population of malignant cells. The method of the invention involves depleting fibroblasts from a population of cells obtained from a tissue sample; selecting epithelial cells from the fibroblast-depleted population; and culturing the selected epithelial cell population in the presence of bone marrow stromal cells so that an enriched population of malignant cells is selected. In particular embodiments, an anchorage-independent population of malignant cells is obtained by conducting at least one passage of the selected malignant cells. Enriched populations of malignant cells are also provided.

### DETAILED DESCRIPTION OF THE INVENTION

It has now been found that malignant cells can be selected by depleting fibroblasts from a population of cells obtained from a tissue sample; selecting for epithelial cells in the fibroblast-depleted population; and culturing the epithelial cell-enriched population in the presence of bone marrow stromal cells to expand the malignant cells. The method was found effective and sensitive for selecting primary malignant cells even from needle biopsies. Advantageously, the instant method allows for the selection of subsets of malignant cells with a preference for bone marrow cells. Cells selected by the method of the instant invention are useful in studies to understand the behavior of malignant cells within bone marrow and identifying therapeutics for preventing or treating metastatic cancer.

By way of illustration of the instant method, primary breast tissue cells from surgical samples of more than two dozen breast cancer patients (Stages I-III) were placed in suspension, depleted of fibroblasts, and enriched for epithelial cells. Subsequently, breast cells were either cultured alone in stromal media or added to stromal cultures. Non-malignant cells placed in the stromal media did not survive. In addition, the selected breast cells placed in stromal media alone did not survive. However, those cultured in the presence of bone marrow stromal cells continued to survive until confluent. As a measure of enrichment, cloning efficiencies were determined, i.e., the number of cells in a population which are able to form colonies indicative of a malignant phenotype. Cloning efficiencies of fibroblast-depleted epithelial cells were greater than approximately 75%. For comparison, parallel studies were performed using established breast cancer cell lines. Bone marrow stromal cells were co-cultured with breast cancer cell lines (T47D and MDA-MB-330) or non-tumorigenic cell lines (MCF10A or MDF12A). Non-tumorigenic cell lines did not survive in co-cultures. By the fourth passage, the malignant breast cancer cells were able to be cultured in the absence of bone marrow stroma. Similar morphologies, phenotypes, and presence of cytokeratin were observed in the malignant breast cancer cells and the cell line T47D.

To demonstrate that the cells could be cultured in an anchorage-independent manner, breast cells were assayed in methylcellulose matrix for clonogenic potential. To demonstrate that anchorage-independent cones could be derived from different stages of breast cancer, assays were performed with cells selected from early to late stages of breast cancer. Table 1 shows the number of cells selected from Stages M0, I and III breast cancer patients and the number of malignant breast cancer cells obtained during the expansion process. The results of this analysis indicated that populations of cells enriched for anchorage-independent malignant breast cancer cells could be readily obtained, independent of stage. Similar to cells prior to passage, a cloning efficiency of 82% was achieved with cells from stage M0 patients, whereas cloning efficiencies of 74% and 87% were respectively achieved with cells originally obtained from stage MI and stage MIII patients.

TABLE 1

| | Stage of Breast Cancer | | |
|---|---|---|---|
| | M0 (n = 4) | MI (n = 5) | MIII (n = 10) |
| Starting population | 1012 ± 50 | 5180 ± 32 | 4135 ± 22 |
| Total cells at P4 | 2 × 10⁶ ± 55 | 6 × 10⁶ ± 90 | 5 × 10⁶ ± 66 |
| Fold expansion | 2.0 × 10³ | 1.2 × 10³ | 1.2 × 15 |
| Colonies/10³ | 820 ± 23 | 744 ± 14 | 868 ± 15 |
| Cloning efficiencies | 82% | 74% | 87% |

While malignant cells from different stages were selected, it was noted that co-cultures from tissues taken from Stage M0 breast cancer patients exhibited significantly slower growth rate compared to co-cultures with cells from Stage III breast cancer. Slower growth may have been due to a significantly lower number of malignant cells in the co-cultures or due to slower growing malignant cells in the early stage of breast cancer.

Given the efficiency of selecting malignant cells from surgical tissue samples, it was determined whether malignant cells could be selected and expanded from breast biopsy samples. The results of this analysis indicated that the instant method was sensitive enough to select malignant cells from small tissue samples, including patients with stage M0 breast cancer. Moreover, the method was also sensitive enough to select malignant cells from needle biopsies.

Thus, selection of malignant cells, independent of the stage of cancer or size of the tissue sample, is now possible using the instant method. Malignant cells selected in accordance with the instant method can be used in the characterization of malignant cells from different stages of cancer. Moreover, co-culture of malignant cells with bone marrow stromal cells provides a tool for selecting and expanding malignant cells in culture from small samples for clinical and/or research analyses. An important aspect of the instant method of selecting malignant cells using stromal co-culture is that the method is specifically selecting for malignant cells with a preference for bone marrow. This is significant given the organ-specific potential of distinct cancer cells (e.g., Minn, et al. (2005) J. Clin. Invest. 115:44-55). Thus, this method is useful for obtaining malignant cells for use in research to study differences in malignant cells in various sites of metastasis and identifying therapeutics which target pre-metastatic and or metastatic cells.

Accordingly, the present invention is a method for selecting an enriched population of malignant cells from a tissue sample. For the purposes of the present invention, malignant is used in the conventional sense to describe neoplasms that show aggressive behavior characterized by local invasion or distant metastasis. A tissue sample of use in accordance with the instant method is generally a sample taken from a subject suspected of having or known to have a malignant tumor or mass of cells, wherein the subject is at any stage, i.e., early to late stages, of cancer. The tissue sample can be from any organ including breast, prostate, skin, liver, ovarian, uterine, colon, etc., with particular embodiments embracing breast tissue. Moreover, the tissue sample can be surgical tissue (e.g., mastectomy or lumpectomy tissue, including axillary lymph nodes) or a biopsy sample including fine-needle aspiration, core, and surgical biopsy tissue. Desirably the cells of the tissue sample are separated by physical or chemical means to facilitate removal of normal, healthy cells.

Advantageously, the population of cells selected by the method of the present invention is enriched with malignant cells. Alternatively stated, the result of the instant method is a substantially homogenous population of cells of the same type, e.g., malignant breast cancer cells or prostate cancer cells. As one of skill in the art can appreciate, the enriched population of malignant cells excludes the bone marrow stromal cells of the co-culture. An enriched or substantially homogenous cell population refers to a mixture of cells in which malignant cells constitute more than about 70% of the total number of cells in the population. In particular embodiments, malignant cells constitute approximately, 75%, 80%, 90%, 95%, 97%, 98%, 99% or 100% of the total number of cells in the population. Homogeneity can be readily ascertained based on the presence or absence of cells expressing cell-specific surface marker proteins, e.g., using flow cytometry-based immunophenotyping or immunofluorescence attaining. Alternatively, homogeneity can be determined by morphological or phenotypic characteristics, e.g., the ability of malignant cells to form colonies.

By way of illustration, a determination of the number of fibroblasts present in a population of cells can be carried out by labeling the population of cells with, e.g., one or more fluorescently tagged antibodies which recognize fibroblast-specific cell surface markers. Exemplary fibroblast cell surface markers are well-known in the art and include, but are not limited to, ALCAM, CD34, COL1A1, COL1A2, COL3A1, and PH-4. Labeled cells can then be counted under fluorescent microscopy or by FACS analysis to determine the number of fibroblasts present in the population of cells. As with fibroblasts, the presence or absence of endothelial cells can be identified using well-known endothelial cell surface markers including, but not limited to, ACE, CD14, CD31, CD34, CD105, CDH5, ENG, ICAN2, MCAM, NOS3, PECAM1, PROCR, SELE, SELP, TEK, THBD, VCAM1, and VWF. Epithelial cell surface markers can also be employed, e.g., CD326, CD1D, K6IRS2, KRT10, KRT13, KRT17, KRT18, KRT19, KRT4, KRT5, KRT8, MUC1, TACSTD1.

To select an enriched population of malignant cells, the instant method involves the steps of depleting fibroblasts from a population of cells obtained from a tissue sample; selecting epithelial cells from the fibroblast-depleted population; and culturing the selected epithelial cell population in the presence of bone marrow stromal cells.

A variety of techniques are known and commercially available for depleting fibroblasts from a population of cells. For example, Miltenyi Biotec (Auburn, Calif.) provides Anti-Fibroblast MicroBeads for depletion of fibroblasts from cell cultures or tissue cell preparations. Alternatively, fibroblasts can be removed from the population of cells using one or more of the fibroblast cell surface markers disclosed herein in combination with cell-sorting methods such as immunopanning, FACS, and magnetically labeled beads. Such methods are generally carried out using, e.g., a fluorescently labeled antibody or ligand, which specifically binds to the surface-localized cell marker thereby facilitating sorting of cells expressing said surface-localized cell marker from cells which do not express the marker. Antibodies for use in cell-sorting methods can be obtained from commercial sources or generated using classical cloning and cell fusion techniques well-known to the skilled artisan (see, e.g., Kohler and Milstein (1975) Nature 256:495-497; Harlow and Lane (1988) Antibodies: A Laboratory Manual, Cold Spring Harbor Laboratory, New York).

Subsequent to fibroblast depletion, epithelial cells in the population of cells are identified and selected. As with fibroblast depletion, a variety of techniques are known and commercially available for positively selecting epithelial cells from a population of cells. For example, Miltenyi Biotec (Auburn, Calif.) provides CD31 MicroBead and CD105 MicroBead kits for positive selection of endothelial cells from cell cultures or tissue cell preparations. Alternatively, conventional cell sorting methods can be used in combination with one or more tagged antibodies or ligands which specifically recognize epithelial-specific surface-localized cell markers.

Whether depleting fibroblasts from the population of cells or selecting epithelial cells, each step of the instant method can be repeated one or more times to achieve a more homogenous population of malignant cells. While the resulting population of cells contains both malignant and normal cells, the population of cells at this stage is considered an enriched population of primary malignant cells as fibroblasts and endothelial cells are depleted from the population.

To facilitate the growth and expansion of the enriched population of malignant cells, the population of cells are cultured in the presence of bone marrow stromal cells. Bone marrow stromal cells can be obtained using established methods including FICOLL-HYPAQUE density gradient separation from red blood cells. If additional purity is desired, the cells can be enriched using, e.g., the MACS CD34 isolation kit (Miltenyi Biotec Inc., Sunny Vale, Calif.) or immunoadsorption with a biotinylated anti-CD34 monoclonal antibody. Purity of bone marrow stromal cells can be ascertained by the absence of CD14 and presence of prolyl 4-hydroxylase markers. Moreover, the decrease cell fusion between the malignant cells and the bone marrow stromal cells, the bone marrow stromal cells can be gamma-irradiated prior to use. As such, the stromal cells would remain metabolically active but would not divide.

In accord with the present method, the enriched population of malignant cells and bone marrow stromal cells are mixed at any ratio, with an equal ratio particularly suitable. The population of cells is grown to confluency under suitable media and growth conditions (e.g., as exemplified herein) to expand the enriched population of malignant cells. Advantageously, by expanding this population of cells in the presence of bone marrow stromal cells, subsets of cells which interact with or have a preference for bone marrow stromal cells are selected.

While the expanded and enriched population of malignant cells grown in the presence of bone marrow stromal cells can be used directly in drug screening assays or for research purposes, particular embodiments embrace passaging the enriched population of malignant cells at least one or more times to obtain an anchorage-independent population of malignant cells. As used herein, passaging is intended to mean that the population of cells is grown to confluency, epithelial cells are selected for (e.g., as described above), and the selected epithelial cells are cultured again in the presence of bone marrow stromal cells. In this regard, non-tumorigenic epithelial cells are eliminated. In some embodiments, at least one passage is carried out. In other embodiments, at least two, three, four, or more passages are carried out to achieve an enriched population of anchorage-independent malignant cells.

Malignant cells selected in accordance with the instant method find application in basic research as well as diagnostic and drug discovery assays. Moreover, the method can be used to select resistant cancer stem cells following chemotherapy.

The invention is described in greater detail by the following non-limiting examples.

### EXAMPLE 1

### Reagents and Antibodies

α-Minimum Essential Medium (α-MEM), glutamine and hydrocortisone were purchased from SIGMA (St. Louis, Mo.). Fetal calf sera (FCS) and horse sera (HS) were purchased from HYCLONE Laboratories (Logan, Utah). PE-cytokeratin monoclonal antibody, PE-rat mouse kappa and PE-CD14 monoclonal antibody were purchased from BD Bioscience (San Jose, Calif.). Prolyl-4-hydroxylase monoclonal antibody was purchased from Dako (Glostrup, Denmark). Anti-Epithelial and Anti-Fibroblast were purchased from Miltenyi Biotec (Auburn, Calif.), respectively.

Methylcellulose (1.2%, 4000 centipose, Fisher Scientific) solution was prepared by pouring dry methylcellulose into boiling endotoxin-free double-distilled water and vigorously stirring until the mixture was homogenous. Additional distilled water was added to a final volume of 500 mL and the mixture was cooled to room temperature. Subsequently, 500 mL of 2× Iscove's media (room temperature) was combined with the 500 mL of methylcellulose. Sodium bicarbonate (26.7 mL of a 7.5% solution) was added with stirring in a cold room for 48 hours. The mixture was subsequently aliquoted in 50 mL tubes and stored at −20° C. for at least 2 months prior to use.

### EXAMPLE 2

### Cell Lines

The following cell lines were purchased from American Type Culture Collection (Manasses, Va.): Tumorigenic cell lines, T47D and MDA-MB-330 and non-tumorigenic cell lines, MCF12A and MCF 10A.

### EXAMPLE 3

### Primary Breast Tissue

Excess tissues were taken from samples of surgical procedures. The surgical interventions were done in patients diagnosed with different stages of breast cancer. At the time of surgery, patients were not on any medication. Samples were provided from two sources. The major source of breast tissue was from Brookdale Hospital (Brooklyn, N.Y.). Patient samples were also provided by the Cooperative Human tissue Network. Table 1 shows representative subsets of patients' profile of tissues. Excess samples from needle biopsies were also obtained from Brookdale Hospital.

### EXAMPLE 4

### Cell Suspension from Surgical Breast Tissues

Cells (normal and malignant) were retrieved from breast tissue samples. Cells were separated by either flushing with a 1-cc syringe containing conventional cell culture media, or by dislodging cells with serrated-end forceps. To avoid cell clumps, petri dishes containing cell suspensions were placed at about 30-45 degree angle. As such, large clumps remained at the top and the clump-free cell suspension could be collected in sterile conical tissue culture tubes. Tubes were filled with standard sera-free media and cells were pelleted by centrifugation at 500 g for 10-15 minutes at room temperature. The cellular pellet was resuspended in Ca⁺⁺/Mg⁺⁺-free PBS (pH 7.2).

Fibroblasts were removed using Anti-Fibroblast Microbeads (Miltenyi Biotec). This was achieved by resuspending cells at 10⁷/mL in PBS and adding Anti-Fibroblast Microbeads to the cells. The mixture was incubated at room temperature for 1-2 hours with mixing at 10-minute intervals. Depending on the degree of malignancy in the biopsies, the incubation time was varied. However, a 1-2 hour incubation period provided consistent results. Magnetically-coupled fibroblasts were removed from the cell suspension and an aliquot of cell suspension was used to determine if the negative fraction was devoid of fibroblasts. To do this, cells were labeled with FITC-anti-fibroblast at 1/1000 dilution (final concentration). PE-anti-cytokeratin was also employed and non-specific labelin was determined with a FITC-isotope control. Cells were microscopically examined or analyzed by FACSCAN to identify the presence of fibroblasts. If fibroblasts were detected, the selection process was repeated until labeling for fibroblasts confirmed depletion.

Upon fibroblast depletion, epithelial cells were positively selected for using Human Epithelial Antigen (HEA) Microbeads (Miltenyi Biotec), according to the manufacturer's instructions. Briefly, cells were resuspended at 10⁷/mL in 1× PBS and added to the Anti-Epithelial Microbeads. The mixture was incubated at room temperature for 2 hours with mixing at 10-minute intervals. Magnetically-coupled epithelial cells were selected by removing the negative fraction. The positive fraction was resuspended in media and an aliquot of cells was used to determine, by immunofluorescence, if the positive fraction was pure. To achieve this, cells were labeled with FITC-anti-CD31 at 1/1000 dilution (final concentration). Non-specific labeling was measured with FITC-isotype control. Cells were immediately examined by microscopy or by FACSCAN and if the results showed the presence of endothelial cells, the selection process was repeated until labeling for epithelial cells determined depletion. Upon endothelial cell depletion, epithelial cells were plated in a standard breast cancer cell media.

Viability of selected cells was >95%, by trypan blue exclusion. Biopsies from three different patients with stage 0 breast cancer (Table 1) were obtained and also cleared of fibroblasts as above. At this stage of selection, fibroblast-depleted cells from patients with breast cancer contained both malignant and normal cells. To select for anchorage-independent cell, the cells were generally passaged at least four times before use in assays. Cryopreservation up to two years did not alter their anchorage-independent properties, nor their ability to form co-cultures.

### EXAMPLE 5

### Preparation of Bone Marrow Stroma

Bone marrow aspirates were obtained from healthy donors between the ages of 18-25. The aspirate was obtained in a syringe containing preservative-free heparin at 50 U/mL. The heparin was diluted in tissue culture media containing 50 U/mL penicillin and 0.05 mg/mL streptomycin. The number of nucleated cells in the aspirates was counted. Approximately 10⁷ nucleated bone marrow aspirate cells were added to a 25-cm² tissue culture flask (FALCON 3109) and the total volume was adjusted to 7 mL with Stroma-I media (α-MEM containing 12.5% fetal bovine serum, 12.5% horse sera, 0.1% μM hydrocortisone, 0.1 μM 2-mercaptoethanol, and 1.6 mM glutamine). Subsequently, the tissue culture flasks were incubated in a 37° C. incubator with 5% CO₂. At day 3, the non-adherent cells were removed from the flasks and placed in a conical tissue culture tube. The cells of each donor were combined. To avoid drying, 6 mL of stromal media was quickly added to the tissue culture flasks and the flasks were returned to the culture incubator. Subsequently, tubes containing the non-adherent cells were centrifuged at 500 g. Generally, when the total volume was ˜50 mL, the tubes were centrifuged for 20-30 minutes. When the total volume was 10-20 mL, the tubes were centrifuged for 10-15 minutes. Media was aspirated and the pellet was resuspended in sera-free α-MEM. In general, pellets from five flasks were resuspended in 20 mL α-MEM.

Cells were separated by adding an equal volume of FICOLL HYPAQUE to the bottom of each tube followed by centrifugation at room temperature for 25-30 minutes at 500 g. The top layer containing the suspension media was aspirated and the next layer containing the bone marrow mononuclear fraction was aspirated and transferred into a clean sterile conical tissue culture tube. The cells were resuspended with 10-20 volumes of sera-free α-MEM and centrifuged at 500 g for 20-30 minutes at room temperature. Medium was aspirated from the pellet and the pellet was resuspended in stroma-I media. Generally, when the starting number of flasks was three, 3 mL of stromal media was added. Approximately 1 mL of cell suspension was added to a pre-warmed tissue culture flask and the flasks were incubated in a 37° C. incubator with 5% CO₂. Each week, 50% of the culture media was replaced with fresh stromal media until confluent.

Confluent stromal cells were trypsinized by adding to each flask 1-2 mL of 0.05% trypsin with 0.053 Na-EDTA. The cells were incubated in a 37° C.-incubator for 5 minutes and subsequently examined with an inverted microscope. De-adhered cells from a particular donor were collected, pooled and placed into a conical tissue culture tube containing α-MEM with 10% FCS. Cells were pelleted by centrifugation at 500 g for 10-15 minutes. Trypsin-sensitive cells were collected and placed in a test tube containing standard tissue culture media with 10% FCS. Cells were fully resuspended in stroma-II media (α-MEM with 20% heat inactivated FCS). One mL of cell suspension was added to a 25-cm² tissue culture flask and the volume was adjusted to 7 mL with fresh stroma-II media. At confluence, the cells were again trypsinized, pelleted, resuspended in stroma-II media as above and grown to confluency, approximately 3 weeks. Passaging in this manner was repeated four times, i.e., adherent cells were passed at least five times.

Purity of the bone marrow stromal cells was confirmed by immunofluorescence using labeled anti-CD14 antibodies (PE, FITC or any other fluorochrome) and labeled anti-fibroblasts antibodies, wherein the labels used for each antibody were non-overlapping fluorochromes. For example, if anti-CD14 antibody was conjugated to PE, anti-fibroblast antibody was conjugated to any fluorochrome but PE. Bone marrow stromal cell preparations were considered pure when they were negative for CD14 and positive for fibroblasts (i.e., prolyl 4-hydroxylase positive).

### EXAMPLE 6

### Co-Cultures with Established Cell Lines

Equivalent numbers of breast cancer cells and bone marrow stromal were co-cultured in stromal growth media with weekly replacement of 50% of the culture media. For cultures in 25-cm² tissue culture flasks, cultures were initiated with 10² cells of each cell subset. To perform timeline studies, at different times, cells are trypsinized and the breast cancer cell lines were positively selected by subjecting the cells twice to positive selection with DYNABEAD conjugated anti-cytokeratin, according to established methods (Rameshwar, et al. (2001) J. Neuroimmunol. 121:22-31). Both stromal and breast cancer cell line populations were counted and the purity of each was verified by flow cytometry with epithelial and fibroblasts antibodies. For epithelia, cells were labeled with PE-cytokeratin monoclonal antibody. Indirect labeling was done from stroma, first and prolyl 4-hydroxylase monoclonal antibody followed with PE-rat anti-mouse IgG. Cells separated by this procedure showed purity of more than ninety nine percent.

### EXAMPLE 7

### Selecting Malignant Breast Cancer Cells from Fibroblast-Depleted Cultures

Epithelial cells retrieved from surgical breast tissues, 10³-10⁶, were added to 25-cm² tissue culture flasks with 7 mL of stromal media and stromal cells at 20-40% confluence. As such, approximately equal numbers of bone marrow stromal cells and fibroblast-depleted epithelial breast cells were combined in flasks containing stromal media II. Cells were grown to confluency and during that time, 50% of the media was replaced weekly. Malignant breast cancer cells were selected using the positive selection approach described herein for obtaining epithelial cells. After selection, co-cultures were reestablished with fresh stroma and the selected epithelial cells. The process of selecting and co-culturing was repeated up to four times. Cells were then studied for cloning efficiency in methylcellulose matrix. If the efficiency was <90%, the selection process was repeated until cloning efficiencies were >90%.

### EXAMPLE 8

### Clonogenic Assays

Clonogenic assays were performed according to established methods (Rao, et al. (2004) supra). Briefly, 10³ cells/mL were resuspended in 1.2% methylcellulose containing media taken from the co-cultures. One mL of cell suspension was added to 35-mm suspension dishes (NUNC) and the cultures were incubated for 1 week at 37° C. Colonies with >15-25 cells were counted and the cloning efficiency presented as percent of total cells placed in culture.

### EXAMPLE 9

### Immunofluorescence Staining

Cells were collected in a polypropylene test tube, and centrifuged for 10 minutes at 150 rpm. The pellet was resuspended in 1× PBS and cells were again pelleted for 10 minutes at 150 rpm. PBS was aspirated and the cell pellet was resuspended in 1-2 mL PBS. Primary antibodies, i.e. anti-CD14 and anti-fibroblast (1:1000 dilution) were added to the cells and the mixture was incubated for 1-2 hours with shaking every 10 minutes. The cells were washed 2 times with 1× PBS and readings were taken on a flow cytometer. If CD-14-positive cells were detected, passaging was repeated.

What is claimed is:

1. A method for selecting an enriched population of malignant cells comprising
depleting fibroblasts from a population of cells obtained from a tissue sample;
selecting epithelial cells from the fibroblast-depleted population; and
culturing the selected epithelial cell population in the presence of bone marrow stromal cells so that an enriched population of malignant cells is selected.

2. The method of claim 1, further comprising conducting at least one passage of the selected malignant cells to obtain an anchorage-independent population of malignant cells.

3. An enriched population of malignant cells selected by the method of claim 1.

4. An anchorage-independent population of malignant cells selected by the method of claim 2.
